(19) United States
(12) Patent Application Publication
(10) Pub. No.: US 2008/0090788 A1
(43) Pub. Date: Apr. 17, 2008

(54) Chemical Compounds

(76) Inventors: Graham Keith Packham, Southampton (GB); Arasu Ganesan, Southampton (GB); Derek A. Mann, Southampton (GB); Fiona Natarajan, Southampton (GB); Srinvian Natarajan, Southampton (GB)

Correspondence Address: NIXON & VANDERHYE, PC, 901 NORTH GLEBE ROAD, 11TH FLOOR, ARLINGTON, VA, 22203, US

(21) Appl. No.: 11/794,711

(22) PCT Filed: Jan. 5, 2006
(86) PCT No.: PCT/GB06/00018

§ 371 Date: Sep. 11, 2007

(30) Foreign Application Priority Data
Jan. 7, 2005 (GB) 0500301.7

(51) Int. Cl. A61K 31/63; A61K 31/635; A61K 31/655; A61P 29/00; A61P 31/00; A61P 35/00; C07C 311/38; C09B 29/00

(52) U.S. Cl. 514/150; 514/155; 514/156; 534/660; 534/664; 534/843; 546/312; 564/86

(57) ABSTRACT

Use of a compound of formula (I), or a pharmaceutically acceptable salt thereof, in the manufacture of a medicament for use as a Nuclear Factor-kB (NF-kB) inhibitor 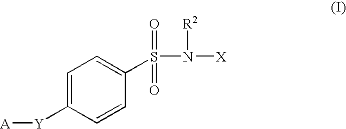 wherein:
A has the following structure; 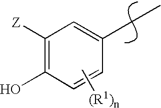
Z is —COOH, —P(O)(OH)₂ or —SO₂OH;
each R¹ is the same or different and is halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl; n is 0, 1, 2 or 3;
R² is hydrogen, C₁₋₆ alkyl, C₂₋₆ alkenyl or C₂₋₆ alkynyl; Y is a linking group; and
X is C₁₋₆ alkyl, C₂₋₆ alkenyl, C₂₋₆ alkynyl, aryl, heteroaryl, carbocyclyl, heterocyclyl, -M-aryl, -M-heteroaryl, -M-carbocyclyl or -M-heterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ is as defined above, provided that:
   when n is 0, Z is —COOH, R² is hydrogen and Y is —N═N—, X is other than 2-pyridyl.

The present relation relates to a series of sulfasalazine analogues which are useful as Nuclear Factor-kB (NF-kB) inhibitors.

The NF-kB transcription factor plays a key role in the regulation of apoptosis by modulating expression of a wide range of cell death control molecules, including adhesion molecules, cytokines, and growth and survival factors. 1-3 NF-kB is composed of heterodimeric or homodimeric complexes of Re1 family proteins, c-Re1, Re1A (p65), Re1B, p50 (NF-kB1) and p52 (NF-kB2). The commonest form of NF-kB is the p65-p50 heterodimer.

NF-kB exists in many cells as a latent form, inactivated by binding to inhibitor proteins (such as inhibitor of kB-α (IkB-α)) in the cytoplasm.

A wide variety of signals, including tumour necrosis factor-α and lipopolysaccharide (LPS), activate a kinase complex, termed IkB kinase complex (IKK), which phosphorylates IkB-α on serines 32 and 36, triggering the ubiquitin-mediated degradation of IkB-α.

IkB-α degradation results in the release and transport of NF-kB to the nucleus where it binds to consensus DNA sequences in the promoter regions of target genes. Altered NF-kB function has been implicated in many human diseases. NF-kB also plays an important role in human diseases by promoting inappropriate cell survival.

Sulfasalazine (SFZ) is a synthetic anti-inflammatory comprising an aminosalicylate, 5-amino salicylic acid (5-ASA), linked to an antibiotic, sulfapynidine (SPY). SFZ, but not 5-ASA or SPY, inhibits activation of NF-kB. SFZ has been shown to inhibit NF-kB activation via direct inhibition of IKK.

It has now been surprisingly found that the sulfasalazine derivatives of general formula (I) shown below inhibit NF-kB.

Accordingly, the present invention provides, in a first embodiment, the use of a compound of formula (I), or a pharmaceutically acceptable salt thereof, in the manufacture of a medicament for use as a Nuclear Factor-kB (NF-kB) inhibitor 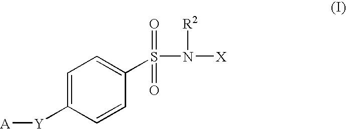
wherein:

A has the following structure;

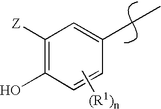

Z is —COOH, —P(O)(OH)₂ or —SO₂OH;

each R¹ is the same or different and is halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl;

n is 0, 1, 2 or 3;

R² is hydrogen, C₁₋₆ alkyl, C₂₋₆ alkenyl or C₂₋₆ alkynyl;

Y is a linking group; and

X is C₁₋₆ alkyl, C₂₋₆ alkenyl, C₂₋₆ alkynyl, aryl, heteroaryl, carbocyclyl, heterocyclyl, -M-aryl, -M-heteroaryl, -M-carbocyclyl or -M-heterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ is as defined above,

provided that:

when n is 0, Z is —COOH, R² is hydrogen and Y is —N═N—, X is other than 2-pyridyl.

For the avoidance of doubt, when the group X is -Q-Het-Q′- or -Q-Het-, the orientation of the group Het is such that the left hand side of the depicted moiety is attached to Q. Thus, for example, when Het is —C(O)—O—, the group -Q-Het-Q′- is -Q-C(O)—O— Q′-. Further, when the group X is -Q-Het-Q′- or -Q-Het-, the Q moiety is attached to the nitrogen atom, N, depicted in formula (I).

As used herein, a C₁₋₆ alkyl group or moiety is a linear or branched alkyl group or moiety containing from 1 to 6 carbon atoms, such as a C₁₋₄ alkyl group or moiety. Examples of C₁₋₄ alkyl groups and moieties include methyl, ethyl, n-propyl, i-propyl, n-butyl, i-butyl and t-butyl. A preferred C₁₋₆ alkyl group is methyl. A C₁₋₆ alkyl group or moiety is unsubstituted or substituted by 1, 2 or 3, for example 1 or 2, substituents selected from halogen, hydroxy, thio or amino, wherein the said substituents are the same or different. Fluorine, chlorine and bromine are preferred substituents and fluorine and chlorine are particularly preferred. An example of a substituted alkyl group is trifluoromethane. A divalent alkyl group (or alkylene group) can be attached via the same carbon atom, via adjacent carbon atoms or via non-adjacent carbon atoms.

As used herein, a C₂₋₆ alkenyl group or moiety is a linear or branched alkenyl group or moiety containing form 2 to 6 carbon atoms, such as a C₂₋₄ alkenyl group or moiety, for example ethenyl, n-propenyl or n-butenyl. A preferred C₂₋₆ alkenyl group is ethenyl. A C₂₋₆ alkenyl group or moiety can be unsubstituted or substituted at any position. Typically, it is unsubstituted or substituted by 1, 2 or 3, for example 1 or 2, substituents, wherein the said substituents are the same or different. Suitable substituents include halogen, hydroxy, thio or amino. Typically an alkenyl group has only one double bond. A divalent alkenyl group (or alkenylene group) can be attached via the same carbon atom, via adjacent carbon atoms or via non-adjacent carbon atoms.

As used herein, a C₂₋₆ alkynyl group or moiety is a linear or branched alkynyl group or moiety containing form 2 to 6 carbon atoms, such as a C₂₋₄ alkynyl group or moiety. A C₂₋₆ alkynyl group or moiety can be unsubstituted or substituted at any position. Typically, it is unsubstituted or substituted by 1, 2 or 3, for example 1 or 2, substituents, wherein the said substituents are the same or different. Suitable substituents include halogen, hydroxy, thio or amino. Typically an alkynyl group has only one triple bond. A divalent alkynyl group (or alkynylene group) can be attached via the same carbon atom, via adjacent carbon atoms or via non-adjacent carbon atoms.

As used herein, an aryl group is typically a C₆₋₁₀ aryl group such as phenyl or naphthyl. Phenyl is preferred. An aryl group may be unsubstituted or substituted at any position. Suitable substituents include halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl. Preferred substituents are hydroxy, C₁₋₆ alkyl or C₁₋₆ alkoxy. More preferred substituents are hydroxy, C₁₋₄ alkyl and C₁₋₄alkoxy. Most preferably, the substituents are selected from C₁₋₂ alkyl and C₁₋₂ alkoxy. Typically the aryl group carries 0, 1, 2 or 3 substituents wherein the said substituents are the same or different. Preferably it carries 0 or 1 substituents. More preferably it carries 1 substituent. Typically, when the aryl group is a phenyl which carries one substituent, the single substituent is at the para position.

As used herein, a heteroaryl group is typically a 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, pyrrolyl, oxadiazolyl, oxazolyl, isoxazolyl, thiadiazolyl, thiazolyl, isothiazolyl, imidazolyl and pyrazolyl groups. Pyridyl is particularly preferred. Typically a heteroaryl group carries 0, 1, 2 or 3 substituents wherein said substituents are the same or different. Suitable substituents include halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl. Preferred substituents are hydroxy, C₁₋₆ alkyl or C₁₋₆ alkoxy. More preferred substituents are hydroxy, C₁₋₄ alkyl and C₁₋₄ alkoxy. Most preferred substituents are C₁₋₂ alkyl and C₁₋₂ alkoxy.

As used herein, a carbocyclyl group is a non-aromatic saturated or unsaturated monocyclic hydrocarbon ring, typically having from 3 to 6 carbon atoms. Preferably it is a saturated hydrocarbon (i.e. a cycloalkyl group) having from 3 to 6 carbon atoms. Examples include cyclopropyl, cyclobutyl, cyclopentyl and cyclohexyl.

A cycloalkyl group may be unsubstituted or substituted at any position. Typically, it carries 0, 1, 2 or 3 substituents wherein said substituents are the same or different. Suitable substituents include halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl. Preferred substituents are hydroxy, C₁₋₆ alkyl or C₁₋₆ alkoxy. More preferred substituents are hydroxy, C₁₋₄ alkyl and C₁₋₄ alkoxy. Most preferred substituents are C₁₋₂ alkyl and C₁₋₂ alkoxy.

As used herein, a heterocyclyl group is a non-aromatic saturated or unsaturated carbocyclic ring typically having from 5 to 10 carbon atoms, in which one or more, for example 1, 2 or 3, of the carbon atoms is replaced by a heteroatom selected from N, O and S. Saturated heterocyclyl groups are preferred.

Examples include tetrahydrofuranyl, tetrahydrothienyl, pyrrolidinyl, imidazolidinyl, pyrazolidinyl, dioxolanyl, thiazolidinyl, tetrahydropyranyl, piperidinyl, dioxanyl, piperazinyl, morpholinyl, thiomorpholinyl, thioxanyl, dithiolanyl, oxazolidinyl, tetrahydrothiopyranyl and dithianyl. Typically, it carries 0, 1, 2 or 3 substituents wherein said substituents are the same or different. Suitable substituents include halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl. Preferred substituents are hydroxy, C₁₋₆ alkyl or C₁₋₆ alkoxy. More preferred substituents are hydroxy, C₁₋₄ alkyl and C₁₋₄ alkoxy. Most preferred substituents are C₁₋₂ alkyl and C₁₋₂ alkoxy.

As used herein, a C₁₋₆ alkoxy group is typically a said C₁₋₆ alkyl group attached to an oxygen atom.

As used herein, a C₁₋₆ alkylthio group is typically a said C₁₋₆ alkyl group attached to a thio group.

As used herein, a halogen is typically chlorine, fluorine, bromine or iodine. It is preferably chlorine, fluorine or bromine. It is more preferably chlorine or fluorine.

Typically, in the compound of formula (I), Z is —COOH.

Typically, in the compound of formula (I), n is 0, 1 or 2. Preferably, n is 1 or 2. More preferably, n is 1.

Typically, R′ is hydrogen or C₁₋₄ alkyl. Preferably, R′ is hydrogen.

Typically, in the compound of formula (I) each R¹ is the same or different and is selected from halogen, hydroxy, C₁₋₆ alkyl, C₂₋₄ alkenyl, C₂₋₄ alkynyl, C₁₋₄ alkoxy, C₁₋₄ alkylthio, thio, amino, mono(C₁₋₄ alkyl)amino, di(C₁₋₄ alkyl)amino and CO₂R′, wherein R′ is as defined above. Preferably, each R¹ is the same or different and is selected from halogen, hydroxy, C₁₋₆ alkyl, C₁₋₄ alkoxy, C₁₋₄ alkylthio, thio, amino or CO₂R′, wherein R′ represents hydrogen or C₁₋₄ alkyl. More preferably, each R¹ is the same or different and is C₁₋₆ alkyl, for example C₁₋₄ alkyl. Preferably, when R¹ is C₁₋₆ alkyl, it is unsubstituted, for example an unsubstituted C₁₋₄ alkyl group.

Preferably, when n is 1, 2 or 3, one R¹ group is positioned meta to the Y group of formula (I) (i.e. at the meta position which does not carry group Z). Preferably, the R¹ group positioned meta to the Y group of formula (I) is C₁₋₆ alkyl. More preferably, it is unsubstituted C₁₋₆ alkyl. Most preferably it is methyl. In a preferred embodiment, n is 1, Z is COOH and the group A is a 2-hydroxy-3-methyl-5-yl-benzoic acid moiety.

Typically, in the compound of formula (I), R² is hydrogen or C₁₋₄ alkyl, C₂₋₄ alkenyl or C₂₋₄ alkynyl. Preferably it is hydrogen or unsubstituted C₁₋₄ alkyl or unsubstituted C₂₋₄ alkenyl. More preferably, R² is hydrogen.

In the compound of formula (I), Y is a linking group. Typically, Y is a divalent group. Preferably Y is —N═N— or C₁₋₆ alkylene, C₂₋₆ alkenylene or C₂₋₆ alkynylene, —C(O)—NR′—, —NR′—C(O)—, -L-Het-L′-, -L-Het- or -Het-L′-, wherein L and L′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O)—, —CO—, —C(O)—NR′— or —NR′—C(O)— wherein R′ is as defined above.

For the avoidance of doubt, when the group Y is -L-Het-L′- or -L-Het-, the orientation of the group Het is such that the left hand side of the depicted moiety is are attached to L. Thus for example, when Het is —C(O)—O—, the group -L-Het- is -L-C(O)—O—. When the group Y is -Het-L′-, the orientation of the group Het is such that the right hand side of the depicted moiety is attached to L′. Thus for example, when Het is —C(O)—O—, the group -Het-L′- is —C(O)—O-L′. Further, when the group Y is —C(O)—NR′—, —NR′—C(O)—, -L-Het-L′-, -L-Het- or -Het-L′-, the left hand side of the group as depicted is attached to A. Thus, for example, when Y is -L-Het-L′-, L is attached to A.

Typically, L and L′ are the same or different and are C₁₋₄ alkylene, C₂₋₄ alkenylene or C₂₋₄ alkynylene.

Het is preferably selected from —NR′—, —C(O)—NR′—, —O—, —S— or —CO—, wherein R′ is as defined above. More preferably, Het is selected from —NR′—, —C(O)—NR′— or —CO—, wherein R′ is hydrogen.

More preferably, Y is —N═N— or C₁₋₄ alkylene, C₂₋₄ alkenylene or C₂₋₄ alkynylene, —C(O)—NR′—, -L-Het-L′-, -L-Het- or -Het-L′- wherein R′, L, L′ and Het are as defined above. More preferably, Y is —N═N— or C₁₋₃ alkylene, C₂₋₃ alkenylene, C₂₋₃ alkynylene, -L-Het- or -Het-L′- wherein Het, L and L′ are as defined above. Yet more preferably, Y is —N═N— or C₁₋₃ alkylene, C₂₋₃ alkenylene, C₂₋₃ alkynylene, -L-Het- or -Het-L′- wherein L and L′ are the same or different and are C₁₋₃ alkylene and Het is —CO—, —NR′— or —C(O)—NR′— wherein R′ is hydrogen or C₁₋₄ alkyl. Preferably the alkylene, alkenylene and alkynylene chains of Y are unsubstituted. It is further preferred that when Y is -L-Het-, L is C₁₋₃ alkylene and Het is —CO—, —NR′— or —C(O)—NR′— wherein R′ is hydrogen or C₁₋₄ alkyl. Yet more preferably, Y is —N═N— or Y is —C≡C—, —C═CH—, —CH₂—CH₂—, —CO—CH═CH—, —CH═CH—CO— or —CH₂—CO—. In a preferred embodiment, Y is —N═N—.

Typically, Q and Q′ are the same or different and are C₁₋₄ alkylene, C₂₋₄ alkenylene or C₂₋₄ alkynylene.

Typically, M is C₁₋₆ alkylene, C₂₋₄ alkenylene, C₂₋₄ alkynylene, -Q-Het-Q′- or -Q-Het- wherein Q, Q′ and Het are as defined above. Preferably, M is C₁₋₆ alkylene, C₂₋₄ alkenylene, C₂₋₄ alkynylene, -Q-Het-Q′- or -Q-Het- wherein Q and Q′ are the same or different and are C₁₋₄ alkylene, C₂₋₄ alkenylene or C₂₋₄ alkynylene and wherein Het is —NR′—, —O—, —S— or —CO—. More preferably, M is C₁₋₆ alkylene, -Q-Het-Q′- or -Q-Het-, wherein Q and Q′ are the same or different and are C₁₋₄ alkylene and Het is —NR′—, —O—, —S— or —CO—. More preferably, M is C₁₋₆ alkylene. Most preferably, M is C₁₋₄ alkylene, for example C₁₋₂ alkylene. Yet more preferably, M is methylene.

Typically in the compound of formula (I), X is C₁₋₆ alkyl, C₂₋₄ alkenyl, C₂₋₄ alkynyl, aryl, heteroaryl, carbocyclyl, heterocyclyl, -M-aryl, -M-heteroaryl, -M-carbocyclyl or -M-heterocyclyl wherein M is as defined above. More preferably X is C₁₋₆ alkyl, aryl, heteroaryl, -M-aryl or -M-heteroaryl, wherein M is as defined above.

More preferably, X is C₁₋₄ alkyl, phenyl, 5- or 6-membered heteroaryl, -M-phenyl or -M-(5- or 6-membered heteroaryl) wherein M is C₁₋₂ alkylene. Preferably the alkylene, alkenylene and alkynylene groups of X are unsubstituted.

Preferred examples of X include substituted or unsubstituted pyridyl, pyrazinyl, pyrimidinyl, pyridazinyl, isoxazolyl or benzyl. When X is pyridyl, it is preferably 2-pyridyl. Particularly preferred examples of X include 2-pyridyl, p-methoxy benzyl and p-trifluoromethyl benzyl.

Typically in formula (I), when R² is hydrogen, Y is —N═N— and A is 2-hydroxy-5-yl-benzoic acid, X is other than 2-hydroxy-4-yl-benzoic acid or 2-hydroxy-5-yl-benzoic acid. Preferably in formula (I), when R² is hydrogen, Y is —N═N— and A is 2-hydroxy-5-yl-benzoic acid, X is other than substituted phenyl and more preferably when R² is hydrogen, Y is —N═N— and A is 2-hydroxy-5-yl-benzoic acid, X is other than unsubstituted or substituted phenyl.

In a preferred embodiment, the compound of formula (I) is not a compound wherein:
A is 2-hydroxy-3-methyl-5-yl benzoic acid, Y is —N═N—, R² is hydrogen and X is 2-pyridyl; or
A is 2-hydroxy-4-amino-5-yl benzoic acid, Y is —N═N—, and R² is hydrogen and X is 2-pyridyl.

In a preferred embodiment, the compound of formula (I) is not 4-fluoro-2-hydroxy-5-[2-[4-[(3-methyl-2-pyridinylamino)sulfonyl]phenyl]ethenyl]benzoic acid.

In a preferred embodiment, the compound of formula (I) is a compound of formula (Ia): 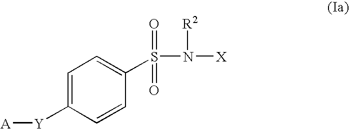
wherein:

A has the following structure;

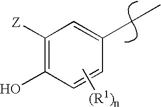

Z, R¹, R², and n are as defined above for formula (I);

Y is —N═N—; and

X is -M-aryl, -M-heteroaryl, -M-carbocyclyl or -M-herterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ is as defined above.

Preferably, in the compounds of formula (Ia), Q and Q′ are the same or different and are selected from C₁₋₄ alkylene, C₂₋₄ alkenylene or C₂₋₄ alkynylene.

Preferably, in the compounds of formula (Ia), M is C₁₋₆ alkylene, for example C₁₋₄ alkylene.

Typically in the compound of formula (Ia), X is -M-aryl, -M-heteroaryl, -M-carbocyclyl or -M-herterocyclyl, wherein M is C₁₋₆ alkylene, C₂₋₄ alkenylene, C₂₋₄ alkynylene, -Q-Het-Q′- or -Q-Het- wherein Q, Q′, and Het are as defined above. More preferably, X is -M-aryl or -M-heteroaryl wherein M is as defined above. Yet more preferably, X is -M-aryl or -M-heteroaryl wherein M is CIA alkylene, for example C₁₋₂ alkylene. Preferably, the alkylene, alkenylene and alkynlene groups of X are unsubstituted. Preferred examples of X include unsubstituted or substituted benzyl. Particularly preferred examples of X are p-methoxy benzyl and p-trifluoromethyl benzyl.

In a ether preferred embodiment, the compound of formula (I) is a compound of formula (Ib): 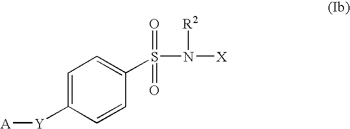
wherein:

A has the following structure;

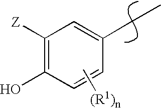

Z, R¹, R² and n are as defined above for formula (I);

Y is C₁₋₆ alkylene, C₂₋₆ alkenylene or C₂₋₆ alkynylene, —C(O)—NR′—, -L-Het-L′-, -L-Het- or -Het-L′-, wherein L and L′ are the same or different and are selected from C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)—, wherein R′ is as defined above;

X is C₁₋₆ alkyl, C₂₋₆ alkenyl, C₂₋₆ alkynyl, aryl, carbocyclyl, -M-aryl, -M-heteroaryl, -M-carbocyclyl or -M-herterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or C(O)— wherein R′ is as defined above.

Typically, in the compound of formula (Ib), L and L′ are the same or different and are C₁₋₄ alkylene, C₂₋₄ alkenylene or C₂₋₄ alkynylene.

Typically, in the compound of formula (Ib), Het is selected from —NR′—, —C(O)—NR′—, —O—, —S—, or —CO—, wherein R′ is as defined above.

In the compound of formula (Ib), Y is preferably C₁₋₆ alkylene, C₂₋₄ alkenylene or C₂₋₄ alkynylene, —C(O)—NR′—, -L-Het-L′-, -L-Het- or -Het-L′- wherein R′, L, L′ and Het are as defined above. More preferably, Y is C₁₋₃ alkylene, C₂₋₃ alkenylene, C₂₋₃ alkynylene, -L-Het or -Het-L′- wherein Het is —CO—, —NR′— or —C(O)—, —NR′—. It is further preferred that when Y is -L-Het-, L is C₁₋₃ alkylene and Het is —CO—, —NR′— or —C(O)—NR′— wherein R′ is hydrogen or C₁₋₄ alkyl. Preferably the alkylene, alkenylene and alkylene chains of Y are unsubstituted. Yet more preferably, Y is —C≡C—, —C═CH—, —CH₂—CH₂—, —CO—CH═CH—, —CH—CH—CO— or —CH₂—CO—.

Typically in the compound of formula (Ib), Q and Q′ are the same or different and are selected from C₁₋₄ alkylene, C₂₋₄ alkenylene or C₂₋₄ alkynylene.

Typically in the compound of formula (Ib), M is C₁₋₆ alkylene, C₂₋₄ alkenylene, C₂₋₄ alkynylene, -Q-Het-Q′- or -Q-Het-, wherein Q, Q′ and Het are as defined above. Preferably in the compound of formula (Ib), M is C₁₋₆ alkylene, C₂₋₄ alkenylene, C₂₋₄ alkynylene, -Q-Het-Q′- or -Q-Het-, wherein Het is —NR′—, —O—, —S— or —CO— and Q, Q′ are as defined above. More preferably M is C₁₋₆ alkylene. Yet more preferably M is C₁₋₄ alkylene, for example methylene.

Typically in the compound of formula (Ib), X is C₁₋₆ alkyl, C₂₋₄ alkenyl, C₂₋₄ alkynyl, aryl, carbocyclyl, -M-aryl, -M-heteroaryl, -M-carbocyclyl or -M-heterocyclyl, wherein M is as defined above. More preferably, X is -M-aryl or -M-heteroaryl, wherein M is as defined above. More preferably, X is -M-aryl or -M-heteroaryl wherein M is C₁₋₄ alkylene, for example C₁₋₂ alkylene. Yet more preferably, X is -M-phenyl or -M-(5- or 6-membered heteroaryl) wherein M is C₁₋₄ alkylene, for example as C₁₋₂ alkylene. Preferably the alkylene, alkenylene and alkylene chains of X are unsubstituted. Preferred examples of X include unsubstituted or substituted benzyl. Particularly preferred examples of X are p-methoxy benzyl and p-trifluoromethyl benzyl.

The present invention includes pharmaceutically acceptable salts of the compound of formula (I). Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutical acceptable bases include alkali metal (e.g. sodium or potassium) and alkaline earth metal (e.g. calcium or magnesium) hydroxides and organic bases such as alkyl amines, aralkyl amines or heterocyclic amines.

Preferred compounds of formula (I) are those wherein:
n is 0, 1 or 2;
each R¹ is the same or different and is halogen, hydroxy, C₁₋₆ alkyl, C₂₋₄ alkenyl, C₂₋₄ alkynyl, C₁₋₄ alkoxy, C₁₋₄ alkylthio, thio, amino, mono(C₁₋₄ alkyl)amino, di(C₁₋₄ alkyl)amino or CO₂R′, wherein R′ is hydrogen or C₁₋₆ alkyl;
R² is hydrogen or C₁₋₄ alkyl;
Z is —COOH;
Y is —N═N— or C₁₋₆ alkylene, C₂₋₆ alkenylene or C₂₋₆ alkynylene, —C(O)—NR′— or —NR′—C(O)—, -L-Het-L′-, -L-Het- or -Het-L′-, wherein L and L′ are the same or different and are C₁₋₆ alkylene, C₂₋₆ alkenylene or C₂₋₆ alkynylene, Het is selected from —NR′—, —O—, —S—, —SO₂—, —SO—, —C(O)—O—, OC(O), —CO—, —C(O)—NR′— or —NR′—C(O)— and R′ is hydrogen or C₁₋₆ alkyl;
X is C₁₋₆ alkyl, C₂₋₄ alkenyl, C₂₋₄ alkynyl, aryl, heteroaryl, carbocyclyl, herterocyclyl, -M-aryl, -M-heteroaryl, -M-carbocyclyl or -M-heterocyclyl, M is C₁₋₆ alkylene, C₂₋₄ alkenylene, C₂₋₄ alkynylene, -Q-Het-Q′- or -Q-Het- and Q and Q′ are the same or different and are C₁₋₄ alkylene, C₂₋₄ alkenylene or C₂₋₄ alkynylene;

and their pharmaceutically acceptable salts.

More preferred compounds of formula (I) are those wherein:
n is 0, 1 or 2;
each R¹ is the same or different and is C₁₋₄ alkyl;
R² is hydrogen;
Z is —COOH;
Y is —N═N—; and
X is C₁₋₄ alkyl, phenyl, 5- or 6-membered heteroaryl, -M-phenyl or -M-(5- or 6-membered heteroaryl), wherein M is C₁₋₂ alkylene,

and their pharmaceutically acceptable salts.

Particularly preferred compounds of the invention include:
2-hydroxy-5-[4-(4-methoxy-benzylsulfamoyl)-phenylazo]-benzoic acid;
2-hydroxy-3-methyl-5-[4-(pyridin-2-ylsulfamoyl)-phenylazo]benzoic acid;
2-hydroxy-5-(4-methylsulfamoyl-phenylazo)-benzoic acid;
2-hydroxy-3-methyl-5-[4-(4-trifluoromethyl-benzylsulfamoyl)-phenylazo]-benzoic acid; and
2-hydroxy-5-[4-(4-trifluoromethyl-benzylsulfamoyl)-phenylazo]-benzoic acid.

Particularly preferred compounds of the invention include:
2-hydroxy-5-[4-(4-methoxy-benzylsulfamoyl)-phenylazo]-benzoic acid;
2-hydroxy-3-methyl-5-[4-(4-trifluoromethyl-benzylsulfamoyl)-phenylazo]-benzoic acid; and
2-hydroxy-5-[4-(4-trifluoromethyl-benzylsulfamoyl)-phenylazo]-benzoic acid

and pharmaceutically acceptable salts thereof.

Compounds of formula (I) may be prepared by the general synthetic route shown below in scheme (a): 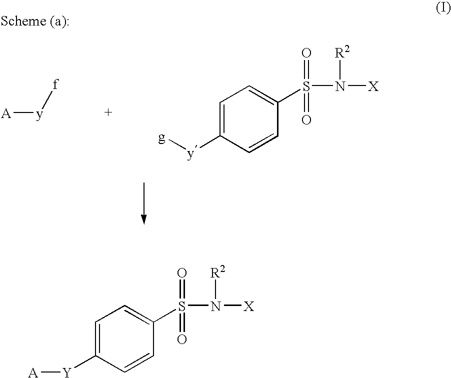
wherein A, Y, R² and X are as defined above, f and g are leaving groups and y and y′ together join to form the species -y-y′-, wherein -y-y′- is Y, as defined above. The starting materials are readily available or can be synthesised by methods known in the art. Similarly, suitable reaction conditions are known in the art.
Compounds of formula (I) wherein Y is —N═N— are typically prepared by coupling species A-H to a diazonium species, as shown in Scheme (b) below: 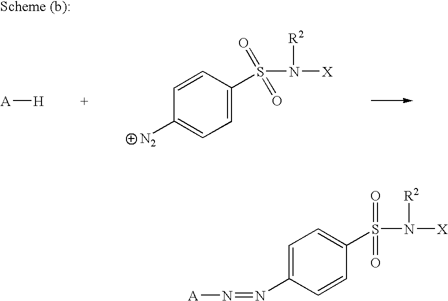
Compounds of formula (I) wherein Y is C₁₋₃ alkylene are typically prepared by Suzuki cross-coupling reactions as shown in Scheme (c) below: 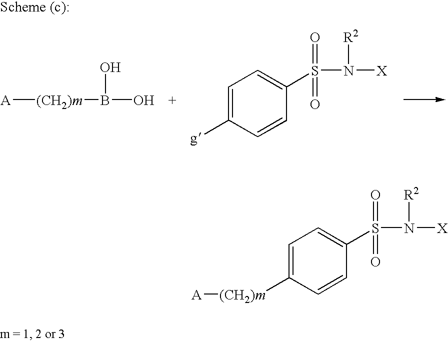
wherein g′ is a halogen atom, for example bromine or iodine.
Compounds of formula (I) wherein Y is a C₁₋₂ alkylamine group are typically prepared by a Buchwald-Hartwig cross-coupling reaction, as shown below in Scheme (d): 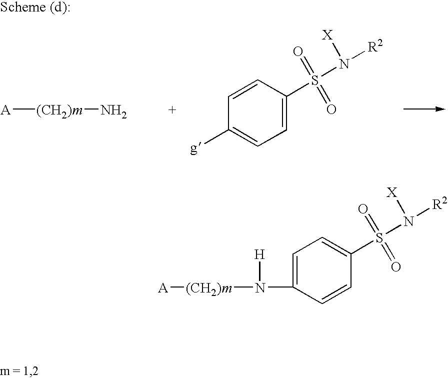
wherein g′ is a halogen atom, for example bromine or iodine.
Compounds of formula (I) wherein Y is a C₁₋₂ amide group are typically prepared by a coupling reaction as shown below in Scheme (e): 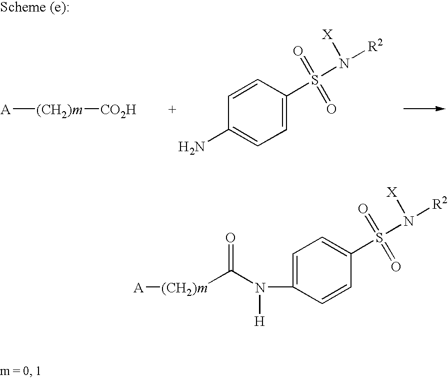
Compound of formula (I) wherein Y is an acetylenic group are typically prepared by a palladium catalysed Sonogashira coupling reaction, as shown below in Scheme (f): 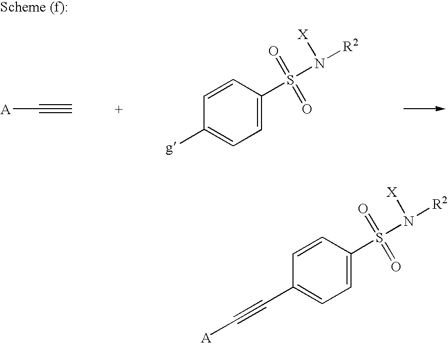
wherein g′ is a halogen atom, for example bromine or iodine.

As explained above, the compounds of the invention are useful as NF-kB inhibitors. Conditions which may be treated or prevented with compounds of the present invention include conditions which are mediated by NF-kB. Examples of conditions which are mediated by NF-kB include conditions which exhibit genetic alteration of NF-kB components and conditions associated with high or increased levels of NF-kB activity. The present invention therefore provides a method for treating a patient suffering from or susceptible to a condition which can be treated with an NF-kB inhibitor, which method comprises administering to said patient an effective amount of a compound of formula (I) or a pharmaceutically acceptable salt thereof.

Examples of conditions which can be treated or prevented with the compounds of the invention include fibrosis, infection, cancer, inflammatory conditions, stroke, myocardial infarction and reperfusion injury.

Examples of fibrosis which can be treated or prevented with the compounds of the invention include fibrosis of the liver, kidney, heart, central nervous system (CNS) lung, skin, airways, pancreas, eye, vascular fibrosis or endometriosis. Preferred examples of fibrosis of the CNS include glial and neural scarring. A preferred example of fibriosis of the skin is scleroderma. A preferred example of fibrosis of the airways is chronic asthma. A preferred example of fibrosis of the eye is macular degeneration. Preferred examples of vascular fibrosis include restonosis and atherosclerosis.

It is particularly preferred that the fibrosis to be treated or prevented with the compounds of the present invention is fibrosis of the liver.

The inflammatory conditions which can be treated by the compounds of the invention include both chronic and acute inflammatory conditions. Examples of the inflammatory conditions include inflammation of the joints and inflammation of the gut. A preferred example of inflammation of the joints is arthritis, particularly rheumatoid arthritis. Preferred examples of inflammation of the gut include inflammatory bowel disease and Crohn's Disease.

Examples of infection which can be treated or prevented with the present compounds include viral, prion and bacterial infection. Preferred examples of viral infections include (i) double stranded DNA viruses, for example adenoviridia, herpesviridia and pappilomaviridia; (ii) single stranded DNA viruses, for example parvoviridia, retroviruses, for example Human Immunodeficiency Virus (HIV) and HTLVI (human T cell lymphotropic virus type I); (iii) negative strand RNA viruses, for example filoviridia, orthomyxovirdia and bunyaviridia; and (iv) positive strand RNA viruses, for example flaviviridia, picornoviridia and coronaviridia. Preferred examples of bacterial infections include (i) gram positive cocci, for example streptococcus and enterococcus; (ii) gram positive rods, for example listeria; (iii) gram negative bacteria, for example salmonella, escherichia and klebsiella; and (iv) gram negative rods, for example campylobactor and helicobactor.

Examples of cancer which can be treated or prevented with the present compounds include malignant tumours. Examples of malignant tumours which can be treated or prevented using the present compounds include diffuse large B-cell lymphoma, follicular large cell lymphoma, follicular lymphoma, mantle cell lymphoma, Epstein-Barr virus associated lymphoma, MALT lymphoma, primary mediastinal lymphoma, multiple myeloma, Hodgkin's disease, chronic lymphocytic leukemia, acute lymphoblastic leukemia (B or T-cell), cutaneous T-cell lymphoma chronic myeloid leukemia, adult T-cell leukemia, childhood acute lymphoblastic leukemia, HTLV-1 associated T-cell leukemia and acute myeloid leukemia. Preferably the malignant tumour is a B-cell malignant tumour selected from diffuse large B-cell lymphoma, follicular large cell lymphoma, follicular lymphoma, mantle cell lymphoma, Epstein-Barr virus associated lymphoma, MALT lymphoma, primary mediastinal lymphoma, multiple myeloma, Hodgkin's disease or chronic lymphocytic leukemia.

It is known that elevated NF-kB activity can promote resistance to some medical therapies. Some therapies may themselves increase NF-kB levels as an unwanted side effect. For example, high levels of NF-kB in cancer cells can promote resistance to therapies used to treat the cancer, for example cytotoxic drug therapy or radiation therapy. Furthermore cytotoxic drug therapy or radiation therapy can in themselves increase NF-kB activity thus limiting the efficacy of these therapies. Sulfasalazine has been shown to sensitise some cancer cells, for example pancreatic carcinoma cells, to cytotoxic drugs, for example etoposide (VP 16) (Int J Cancer 2003 Apr. 20; 104(4): 469-76).

The present invention therefore further provides the use of a compound of formula (I) or a pharmaceutically acceptable salt thereof as defined above in the manufacture of a medicament for reducing the side effects of a therapy, wherein the said therapy increases NF-kB activity. Preferably the said therapy is treatment with a cytotoxic drug, radiation therapy, treatment with an anti-viral drug or treatment with an immunosuppressant drug. Cytotoxic drugs which increase NF-kB activity are known in the art (e.g. Cancer Res 2001 Nov. 1; 61(21):7785-91, J Am Coll Surg 2004 April; 198(4):591-9 and Biochem Pharmacol 2002 May 1; 63(9):1709-16). Preferred examples of cytotoxic drugs which increase NF-kB activity include doxorubicin, SN38, gemcitabine, taxol, 5-fluorouracil and cisplatin.

Preferred examples of an anti-viral drug include ribavarin, PEG-interferon or lamivudine. Preferred examples of immunosuppressant drugs include cyclosporine.

The present invention further provides the use of a compound of formula (I) or a pharmaceutically acceptable salt thereof as defined above in the manufacture of a medicament for reducing, reverting or preventing the development of resistance to a therapy, wherein the said therapy is rendered less active in the presence of NF-kB activity. Preferably the said therapy is a treatment with a cytotoxic drug, radiation therapy, treatment with an anti-viral drug or treatment with an immunosuppressant drug. Typical anti-viral drugs and immunosuppressant drugs are defined above. Examples of cytotoxic drugs wherein NF-kB contributes to their resistance are known in the art (e.g. Cancer Research 2001 Nov. 1; 61(21):7785-91, Int J Cancer 2003 Apr. 20; 104(4):469-76 and Oncogene 2003 May 22; 22(21): 3243-51) and include topoisomerase-2 inhibitors. Preferred examples of cytotoxic drugs wherein NF-kB contributes to their resistance are etoposide (VP16), gemcitabine, doxorubicin and SN38.

The present invention further relates to a combination of a compound of formula (I), or a pharmaceutically acceptable salt thereof, with one or more cytotoxic drugs, an anti-viral drug or an immunosuppressant drug and the use of such a combination as an NF-kB inhibitor. Preferably the combination is used in the treatment of a condition which exhibits increased levels of NF-kB activity. Examples of suitable cytotoxic, anti-viral and immunosurpressant drugs are given above.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

In a preferred embodiment, the compounds of the invention are administered, parenterally. It is particularly preferred that compounds of the present invention wherein Y is a hydrolysable group, for example wherein Y is —N═N— or wherein Y is an amide group, are administered parenterally. The present invention also provides a pharmaceutical composition containing a compound of formula (I), or a pharmaceutically acceptable salt thereof, and a pharmaceutically acceptable carrier or diluent.

Said pharmaceutical composition typically contains up to 85 wt % of a compound of the invention. More typically, it contains up to 50 wt % of a compound of the invention. Preferred pharmaceutical compositions are sterile.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

A therapeutically effective amount of a compound of the invention is administered to a patient. A typical dose is from about 0.001 to 50 mg per kg of body weight, according to the activity of the specific compound, the age, weight and conditions of the subject to be treated, the type and severity of the disease and the frequency and route of administration. Preferably, daily dosage levels are from 5 mg to 2 g.

Certain compounds of the formula (I) are novel per se. The present invention includes these novel compounds and pharmaceutically acceptable salts thereof. The present invention therefore also provides compounds of formula (Ia) and (Ib) as defined above and their pharmaceutically acceptable salts. The present invention further provides compounds of formula (II) and (III), as defined below, and their pharmaceutically acceptable salts.

Compound (II) is a compound of general formula (I) having the general formula as shown below: 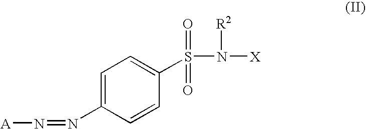
wherein:

A has the following structure

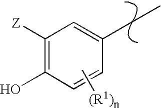

Z, R¹, R² and X are as defined above for formula (I);

n is 1, 2 or 3,

provided that:

when A is 2-hydroxy-3-methyl-5-yl benzoic acid and R² is hydrogen, X is other than 2-pyridyl; and

when A is 2-hydroxy-4-amino-5-yl benzoic acid and R² is hydrogen, X is other than 2-pyridyl.

Preferably, in the compound of formula (II), n is 1 or 2. More preferably n is 1.

Preferably, in the compound of formula (I), X is other than X is other than 2-pyridyl. More preferably in the compound of formula (II), X is other than substituted heteroaryl and yet more preferably X is other than unsubstituted or substituted heteroaryl.

Preferably in the compound of formula (I), X is C₁₋₆ alkyl, C₂₋₆ alkenyl, C₂₋₆ alkynyl, aryl, carbocyclyl, herterocyclyl, -M-aryl, -M-heteroaryl, -M-carbocyclyl or -M-herterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ is hydrogen or or C₁₋₆ alkyl.

Compound (IM) is a compound of formula (I) having the general formula as shown below: 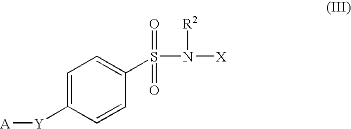
wherein:

A has the following structure

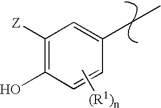

Z, R¹, R² and X are as defined above for formula (I);

Y is C₁₋₆ alkylene, C₂₋₆ alkenylene, C₂₋₆ alkynylene, —C(O)—NR′—, -L-Het-L′-, -L-Het- or -Het-L′-, wherein L and L′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ is as defined above; and

n is 1, 2 or 3;

other than 4-fluoro-2-hydroxy-5-[2-[4-[(3-methyl-2-pyridinylamino)sulfonyl]phenyl]ethenyl]benzoic acid.

Typically in the compound of formula (III), L and L′ are the same or different and are C₁₋₄ alkylene, C₂₋₄ alkenylene or C₂₋₄ alkynylene.

Typically in the compound of formula (III), Het is selected from —NR′—, —C(O)—NR′—, —O—, —S— or —CO—, wherein R′ is as defined above.

Typically in the compound of formula (III), Y is C₁₋₄ alkylene, C₂₋₄ alkenylene or C₂₋₄ alkynylene, -L-Het-L′-, -L-Het- or -Het-L′ wherein L, L′ and Het are as defined above. Preferably, Y is —N═N— or C₁₋₃ alkylene, C₂₋₃ alkenylene, C₂₋₃ alkynylene, -L-Het- or -Het-L′ wherein Het is —CO—, —NR′— or —C(O)—NR′—, L and L′ are the same or different and are C₁₋₄ alkylene, C₂₋₄ alkenylene or C₂₋₄ alkynylene and R′ is hydrogen. Preferably the alkylene, alkenylene and alkynylene chains of Y are unsubstituted. It is further preferred that when Y is -L-Het-, L is C₁₋₃ alkylene and Het is —CO—, —NR′— or —C(O)—NR′—. Yet more preferably, Y is —C≡C—, —C═CH—, —CH₂—CH₂—, —CO—CH═CH—, —CH═CH—CO— or —CH₂—CO—.

Preferably, in the compound of formula (III), n is 1 or 2. More preferably n is 1.

Preferably, in the compound of formula (III), X is other than X is other than 2-pyridyl. More preferably, in the compound of formula (III), X is other than substituted heteroaryl and yet more preferably X is other than unsubstituted or substituted heteroaryl.

Preferably in the compound of formula (III), X is C₁₋₆ alkyl, C₂₋₆ alkenyl, C₂₋₆ alkynyl, aryl, carbocyclyl, heterocyclyl, -M-aryl, -M-heteroaryl, -M-carbocyclyl or -M-heterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ is as defined above.

Typically, in the compound of formula (III) each R¹ is the same or different and is selected from hydroxy, C₁₋₆ alkyl, C₂₋₄ alkenyl, C₂₋₄ alkynyl, C₁₋₄ alkoxy, CIA alkylthio, thio, amino, mono(C₁₋₄ alkyl)amino, di(CIA alkyl)amino and CO₂R′, wherein R′ is as defined above.

Preferably in the compound of formula (III), R¹ is other than halogen, more preferably it is other than fluorine.

Preferred examples of novel compounds of formula (I) are:
2-hydroxy-5-[4-(4-methoxy-benzylsulfamoyl)-phenylazo]-benzoic acid;
2-hydroxy-5-(4-methylsulfamoyl-phenylazo)-benzoic acid;
2-hydroxy-3-methyl-5-[4-(4-trifluoromethyl-benzylsulfamoyl)-phenylazo]-benzoic acid; and
2-hydroxy-5-[4-(4-trifluoromethyl-benzylsulfamoyl)-phenylazo]-benzoic acid,
   and their pharmaceutically acceptable salts.

Particularly preferred examples of novel compounds of formula (I) are:
2-hydroxy-5-[4-(4-methoxy-benzylsulfamoyl)-phenylazo]-benzoic acid;
2-hydroxy-3-methyl-5-[4-(4-trifluoromethyl-benzylsulfamoyl)-phenylazo]-benzoic acid; and
2-hydroxy-5-[4-(4-trifluoromethyl-benzylsulfamoyl)-phenylazo]-benzoic acid,
   and their pharmaceutically acceptable salts.

The present invention also provides the novel compounds of formula (I) for use in the treatment of the human or animal body. The present invention therefore also provides a compound of formula (Ia), (Ib), (II) or (III), or a pharmaceutically acceptable salt thereof, for use in the treatment of the human or animal body.

The present invention also provides a composition comprising a novel compound of formula (I) and a pharmaceutically acceptable carrier. The present invention therefore also provides a composition comprising a compound of formula (Ia), (Ib), (II) or (III), or a pharmaceutically acceptable salt thereof, and a pharmaceutically acceptable carrier.

### EXAMPLES

SFZ was from Calbiochem (Nottingham, UK) and 5-ASA and SPY were from Sigma Aldrich (Poole, UK). Chemical synthesis of SFZ derivatives was carried out according to the general scheme shown in Scheme (1) below. Commercially available p-nitrobenzenesulfonyl chloride was converted to the sulfonamide under Schotten-Baumann conditions, and the nitro group then reduced with iron to yield the amine (Ramadas, K. and Srinivasan, N. Iron-ammonium chloride—a convenient and inexpensive reductant. Synth Commun 1992; 22: 1389-1395). This was diazotised and coupled to the salicylic acid, analogously to literature precedents (Carceller, E., Salas, J., Merlos, M., et al. Novel azo derivatives as prodrugs of 5-aminosalicylic acid and amino derivatives with potent platelet activating factor antagonist activity. J Med Chem 2001; 44: 3001-3013). The structures of compounds 2a-d and 3a-f are shown in Table 1. All compounds were dissolved as stock solutions at 250 mM in DMSO and stored at −20° C. 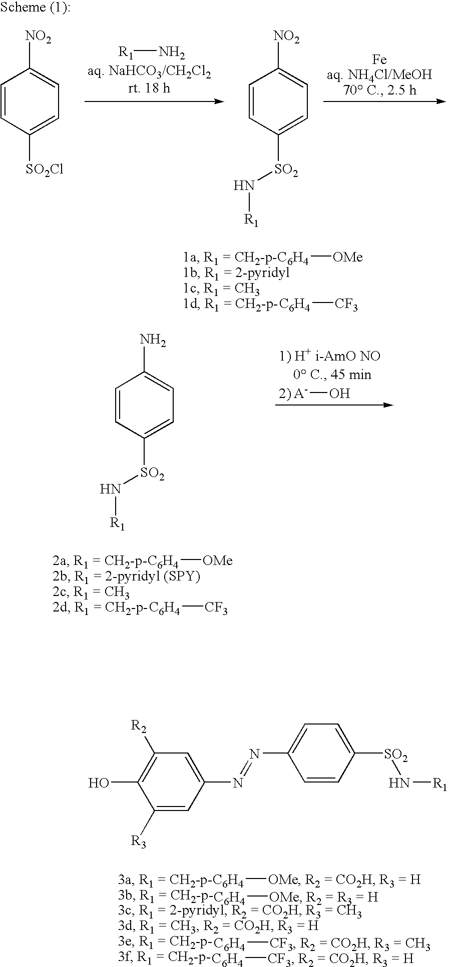
Typical Procedure for Sulfonamide Formation

4-Nitrobenzenesulfonyl chloride (2.0 g, 6.1 mmol) was dissolved in dicholoromethane (20 mL) and cooled to 0-5° C. To this mixture, methylamine (40% aq, 2 mL, 18.3 mmol) was added slowly with stirring. After 20 minutes, the reaction mixture was stirred at room temperature overnight, followed by dilution with water (10 mL). The dichloromethane layer was separated, washed with water and brine, dried (anhydrous MgSO₄), and evaporated to give a yellow solid. Recrystallisation using hexane/dichloromethane provided sulfonamide 1c as a pale yellow crystalline solid (1.03 g, 58%).

Typical Procedure for Nitro Group Reduction

To a 100 mL two-necked RB flask fitted with a reflux condenser, 4-nitro-N-(p-methoxybenzyl)benzene sulfonamide (1.5 g, 4.65 mmol) and methanol (20 mL) were added before adding iron powder (0.78 g, 13.65 mmol). An aqueous solution of ammonium chloride (1.3 g, 23.3 mmol, 20 mL) was added to the above reaction mixture which was heated to 70° C. under gentle stirring for 2.5 h. The reaction mixture was cooled and filtered to remove the inorganic residues and then washed with acetone (3×5 mL). The combined filtrate and the washings were evaporated to leave a white solid to which 50 mL of water was added. The pH of the solution was maintained basic by adding sodium bicarbonate and cooled in ice bath for 15 minutes. The solid obtained was filtered and dried. Rerystallisation with hexane/acetone solvent mixture furnished the pure aminosulfonamide 2a (1.1 g, 84%) as a white, amorphous powder.

Typical Procedure for the Synthesis of the Sulfasalazine Analogues

To N-(p-methoxybenzyl)-4-aminobenzenesulfonamide (2a, 0.292 g, 1 mmol), conc. HCl (0.5 mL) was added followed by the addition of methanol (3 mL) and acetonitrile (3 mL). The reaction mixture was cooled in an ice bath at 0° C., and iso-amylnitrite (134 μL, 1 mmol) was added dropwise for 10 minutes under inert atmosphere. The yellow reaction mixture was stirred for 45 minutes at 0° C. Meanwhile, salicylic acid (0.138 g, 1 mmol) and potassium carbonate (0.689 g, 5 mmol) were mixed in a two-necked RB flask fitted with septa and stopper. To this mixture, water (10 mL) was added and the clear homogenous mixture was degassed by purging with nitrogen for 15 minutes. The resultant degassed solution was cooled in an ice bath (0-5° C.). The amber coloured diazonium salt solution was added to the cooled solution of potassium salicylate through cannula under inert atmosphere. The reaction mixture changed to orange from yellow and was stirred for 1 hour at 0-5° C. At the end of addition of the diazonium salt solution, the pH of the reaction mixture should be around 8-10. After an hour of stirring, the reaction mixture was carefully acidified with 1N HCl with cooling and the resultant reaction mixture was extracted with ether (4×5 mL). The organic extract was washed with water followed by brine and dried over anhydrous MgSO₄. The dried ether extract on evaporation furnished the desired sulfasalazine analogue as a yellow solid. The solid obtained was dissolved in a minimum amount of dichloromethane and cooled, followed by the addition of hexane. This led to precipitation of the desired compound 3a in good purity free from unreacted salicylic acid which is the potential contaminant.

TABLE 1

| Strcuture | Compound Name |
|---|---|
| 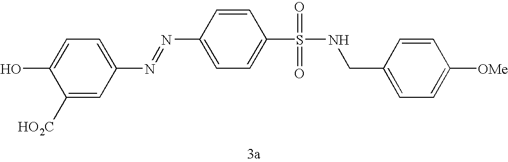 | 2-hydroxy-5-[4-(4-methoxy- benzylsulfamoyl)-phenylazo]- benzoic acid |
| 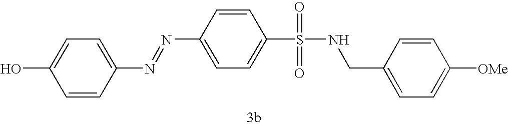 | |
| 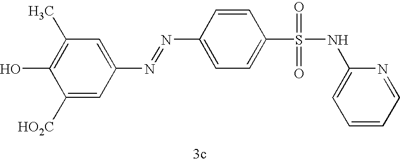 | 2-hydroxy-3-methyl-5-[4- (pyridin-2-ylsulfamoyl)- phenylazo]benzoic acid; |
| 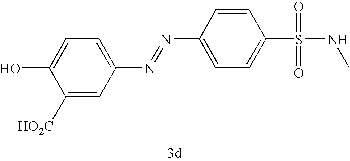 | 2-hydroxy-5-(4- methylsulfamoyl-phenylazo)- benzoic acid; |
| 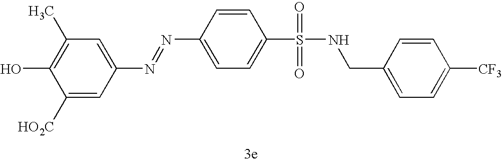 | 2-hydroxy-3-methyl-5-[4-(4- trifluoromethyl- benzylsulfamoyl)-phenylazo]- benzoic acid |
| 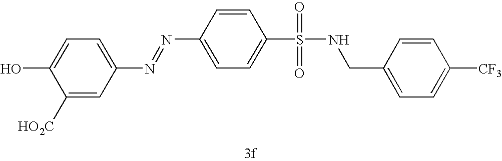 | 2-hydroxy-5-[4-(4- trifluoromethyl- benzylsulfamoyl)-phenylazo]- benzoic acid |
| 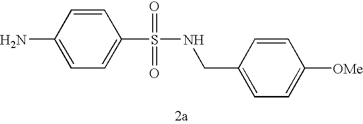 | |
| 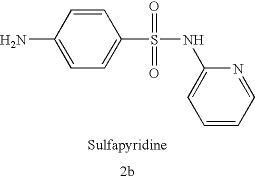 | sulfapyridine |
| 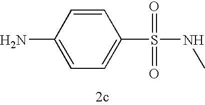 | |
| 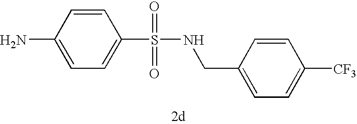 | |

Characterisation Data for Selected Sulfasalazine Analogues:

3a: mp 112-114° C.; IR: 3410 (m), 3334 (m), 3140 (m), 2844 (m), 1680 (m), 1594 (s), 1294 (s), 1138 (s) cm⁻¹; NMR: ¹H (400 MHz, DMSO-d₆, δ ppm) 10.31 (b, 1H, COOH), 8.23 (s, 1H, NH), 7.90-7.40 (m, 1H, Ar), 4.10 (s, 2H, CH₂-Ph), 3.21 (s, 3H, OCH₃); ¹³C (100 MHz, DMSO-d₆, δ ppm) 162.90, 140.61, 132.94, 132.88, 131.13, 129.80, 129.73, 129.43, 129.04, 128.91, 128.66, 127.99, 127.52, 114.40, 53.50, 47.12; ES-MS: m/z (%) 427 ((M+Na⁺), 23).

3e: dark brown dense liquid; IR: 3402 (m), 3329 (w), 2858 (w), 1621 (s), 1596 (m), 1323 (m), 1130 (s), 1064 (s) cm⁻¹; NMR: ¹H (400 MHz, DMSO-d₆, δ ppm) 8.60 (s, 1H, COOH), 8.10 (s, 1H, NH), 7.60-7.2 (m, 1H, Ar), 4.32 (s, 2H, CH₂-Ph); ¹³C (100 MHz, DMSO-d₆, δ ppm) 153.32, 128.44, 125.93, 125.25, 123.01, 120.56, 46.09, 15.84; ES-MS: m/z (%) (361 (M+Na⁺), 100).

3f: dense yellow liquid; IR 2586 (m), 2365 (m), 1740 (s), 1350 (s), 1155 (m) cm⁻; NMR: ¹H (400 MHz, DMSO-d₆, δ ppm) 10.21 (s, 1H, COOH), 7.60-7.30 (m, 11H, Ar), 5.30 (s, 1H, OH), 4.30 (s, 2H, CH₂-Ph); ¹³C (100 MHz, DMSO-d₆, δ ppm) 162.45, 141.17, 133.32, 132.18, 130.81, 129.53, 128.81, 128.32, 128.19, 128.10, 127.65, 127.31, 126.99, 124.99, 122.33, 120.16, 46.37; ES-MS: m/z (%) 517 ((M+Na⁺), 27).

Cells

ARH77 are an Epstein-Barr virus transformed human B-cell line and were maintained in RPMI 1640 medium (Invitrogen Life Technologies, Paisely, UK) supplemented with 10% (v/v) fetal calf serum (FCS) (PAA Laboratories Ltd, Somerset, UK), glutamine and antibiotics (both from Invitrogen Life Technologies). U937-kBluc cells were derived from human U937 histiocytic lymphoma cells (Sundstrom C, Nilsson K. Establishment and characterization of a human histiocytic lymphoma cell line (U-937). Int J Cancer 1976; 17: 565-77) stably transfected with a reporter plasmid containing 3 copies of a consensus NF-kB binding site cloned upstream of a minimal promoter and the luciferase reporter gene. U937-kBluc were maintained in RPMI 1640 medium (Invitrogen Life Technologies) supplemented with 10% (v/v) fetal calf serum (FCS), glutamine and antibiotics. CLL (chronic lymphocytic leukemia) cells were isolated from peripheral blood following informed consent and cryopreserved before use as previously described (Lanham S, et Blood 2003; 101: 1087-93).

Hepatic stellate cells (HSC) were isolated from normal livers of ˜350 g adult male Sprague-Dawley rats by sequential perfusion with collagenase and pronase, followed by discontinuous density centrifugation in 11.5% Optiprep (Invitrogen Life Technologies). HSC were cultured on plastic in Dulbecco's Modified Eagle Medium (DMEM), supplemented with 100 units/ml penicillin, 100 μg/ml streptomycin, 2 mM L-glutamine and 16% (v/v) FCS and maintained at 37° C. at an atmosphere of 5% CO₂. Activated HSC were generated by continuous culture of freshly isolated cells on plastic for 7 days (Wright M C et al Gastroenterology 2001; 121: 685-98).

Analysis of Promoter Activity

U937-kBluc cells were cultured in a 96 well plate (2×10⁴ cells in 100 μl in each well) and stimulated with 0.3 μg/ml LPS (lipopolysaccaride; Sigma Aldrich). After 6 hours, cells were collected and luciferase activity determined using the luclite reagent (Perkin Elmer UK, Beaconsfield, UK) and a Packard TopCount (Perkin Elmer), as described by the manufacturer. To determine the effect of SFZ and analogues, various concentrations of each compound or an equivalent amount of DMSO as a solvent control were added 1 hour prior to addition of LPS. Each determination was made in triplicate.

For HSC transfections, activated HSC were transfected by the non-liposomal Effectene protocol (Qiagen, UK) using 1 μg reporter plasmid DNA and 10 ng of control Renilla plasmid pRLTK (Promega, Southampton, UK) according to the manufacturer's instructions. After 24 hours, HSC were treated with test compounds for a further 24 hours. Luciferase assays were performed using a dual luciferase kit (Promega) according to the manufacturer's instructions. Firefly luciferase activities were normalised for differences in transfection efficiency by measurement of the activity of the co-transfected pRLTK. The pIκBα wt-Luc plasmid contains nucleotides −332 to +35 of the human IkB-α promoter cloned upstream of luciferase gene (Oakley F, et al, J Biol Chem 2003; 278: 24359-70). The pTIMP1-Luc plasmid contains the 162 bp human TIMP1 minimal promoter cloned upstream of luciferase gene (Bertrand-Philippe M, et al. J Biol Chem 2004; 279: 24530-9). The pAP1-Luc plasmid (Stratagene, UK) contains 7 copies of a consensus AP-1 binding site upstream of the minimal promoter and the luciferase gene.

Cell Death/Apoptosis Assays

A flow cytometry method was used to determine the ability of SFZ analogues to induce cell death in ARH77 cells. The proportion of cells that had lost plasma membrane integrity was used as a measure of cell viability. ARH77 (5×10⁴) cells were incubated in a volume of 200 μl in 96 well plates with compound, an equivalent amount of DMSO or were left untreated as a control. After 20 hours, cells were washed in phosphate buffered saline (PBS), resuspended in PBS containing 1 μg/ml propidium iodide (PI) for 10 minutes. Cells were analysed using a FACSCalibur (BD Biosciences, Oxford, UK) using the FL2 channel. Cells which take up PI exhibit intense fluoresence whereas those with intact membranes exclude the dye and have weak fluorescence. The proportion of cells with intense fluorescence as a proportion of total cells was calculated.

CLL apoptosis was quantified by analysing PARP which is cleaved by effector caspases during apoptosis. CLL cells isolated from three individual patients were recovered from storage in liquid nitrogen and cultured at a density of 1.25×10⁶ cells in 1 ml of growth medium (RPMI 1640, 10% (v/v) FCS and antibiotics). After 1 hour, compounds were treated with compounds. Cells were harvested 6 hours after addition of compound, resuspended in 1×SDS-PAGE sample buffer and sonicated prior to immunoblot analysis for PARP expression as described below. PARP cleavage was quantified by determining the amount of cleaved PARP as a percentage of total PARP expression by digital image analysis.

To determine the effects of SFZ analogues on HSC survival, activated HSC were treated for 24 hrs with SFZ or analogues, and apoptotic HSC were visualised by staining with Acridine orange (1 μg/ml in 10 mM 4-(2-hydroxyethyl)-1-piperazineethanesulfonic acid (Hepes) buffer, pH7.4). The proportion of apoptotic cells in five random fields were counted in duplicate wells at x20 magnification using a fluorescein isothiocyanate filter. Apoptosis of HSC measured by acridine orange correlates closely with activation of caspase 3.

Growth Inhibition Assays

To determine the growth inhibitory effects of compounds 2a, SPY, 2c, 2d, ARH77 cells (2×10⁴ cells in 100 μl in each well of a 96 well plate) were incubated with each compound or an equivalent amount of DMSO. Four days later, cell growth was determined using the Aqueous One Solution Cell Proliferation Assay (MTS) (Promega) according to the manufacturer's instructions. Each determination was made in triplicate. Relative cell growth was determined by subtracting the absorbance of wells containing medium and lacking cells, and dividing by absorbance values derived from cells treated with an equivalent volume of DMSO.

Western Blotting

Western blotting was performed as previously described (Brimmell M, Burns J S, Munson P, et al. High level expression of differentially localized BAG-1 isoforms in some oestrogen receptor-positive human breast cancers. Br J Cancer 1999; 81: 1042-51). IkB-α and phospho-Ser32 specific IkB-α antibodies were from Cell Signaling Technology (Hitchin, UK). The PARP antibody (C2-10 clone) was from R&D Systems (Abingdon, UK).

Inhibition of NF-kB Activity

A cell line containing an NF-kB reporter construct was used to determine the effects of SFZ analogues on NF-kB activity. Cells were treated with LPS to induce NF-kB activity in the presence or absence of each compound, or DMSO as a solvent control, and luciferase activity analysed as a measure of NF-kB dependent transcription (FIG. 1). SPY, 5-ASA and compound 3b, lacking the —COOH group in the salicylic acid moiety, inhibited NF-kB activity by <50% at 2 mM and were therefore considered inactive (i.e., IC₅₀>2 mM). Detailed dose response experiments were performed for compounds which resulted in >50% inhibition of NF-kB activity when tested at 2 mM. SFZ inhibited NF-kB activity with an IC₅₀ of 1.194±0.096 mM (mean of duplicate determinations ±SD). Compounds 3c and 3d had mean IC₅₀ values of 1.099±0.040 mM and 1.2380±0.106 mM, respectively, and were not significantly different from SFZ (p>0.3; Student's T-test). The remaining three analogues (3a, 3e and 3f) were significantly better inhibitors of NF-kB activity compared to SFZ (p<0.05) with mean IC₅₀ values of 0.150±0.027 mM, 0.127±0.019 mM, 0.089±0.026 mM, respectively.

To extend these findings, further analyses were performed using the IkB-x promoter which contains multiple NF-kB binding sites and is a well studied NF-kB target. Based on the findings in U937 cells, it was determined whether relatively low doses of analogues 3a, 3e and 3f (i.e., at IC₅₀) were equally effective as a high dose (1 mM) of SFZ for inhibiting transcription in HSC. Consistent with the presence of activated NF-kB in HSC, (Wright M C et al, Gastroenterology 2001; 121: 685-98.) the IkB-α promoter was activated in HSC (FIG. 2a). SFZ (1 mM) inhibited promoter activity by >60%. Importantly, analogues 3a, 3e and 3f, which were more effective inhibitors of NP-kB in LPS-treated U937 cells were approximately equally effective when used at significantly lower concentrations. In contrast, neither SFZ or the analogues interfered with TIMP1 promoter activity, or the activity of an artificial reporter construct containing multiple AP-1 transcription factor binding sites, which are not responsive to NF-kB (FIG. 2 b,c). Therefore, the improved inhibitory activity of analogues 3a, 3e and 3f is specific for NF-kB mediated transcription and is not restricted to U937 cells or artificial promoter constructs.

Effects of SFZ Analogues on IkB-α Phosphorylation

SFZ interferes with the activity of IKK and therefore prevents the phosphorylation and subsequent degradation of IkB-α. (Wahl et al, B. J Clin Invest 1998; 101: 1163-74 and Weber et al, Gastroenterology 2000; 119: 1209-18).

To determine the kinetics of modulation of IkB-α by LPS, U937 cells were treated with LPS for up to 120 minutes and total IkB-α and phosphorylated IkB-αanalysed by immunoblotting. There was a rapid accumulation of phosphorylated IkB-α, accompanied by IkB-α degradation (FIG. 3a). To confirm that the enhanced NF-kB inhibitory activity of compounds 3a, 3e and 3f was due to increased inhibition of IKK, we pretreated cells with these compounds and then stimulated them with LPS. IkB-α was analysed at 30 minutes after the addition of LPS because it was possible to monitor both phosphorylation and degradation at this time. Consistent with inhibition of IKK, compounds 3a, 3e and 3f prevented IkB-α phosphorylation and degradation when used at 500 μM, and were partially effective at 200 μM. Higher doses of SFZ (1 mM) which give partial inhibition of NF-kB activity had only very modest effects on regulation of IkB-α. Therefore, the improved NF-kB inhibitory activity of compounds 3a, 3e and 3f is associated with effective inhibition of IkB-α phosphorylation.

Analysis of Cell Killing in Human Transformed B-Cells

To assess cell death promoting activity, the ability of SFZ and the analogues with enhanced NF-kB inhibiting activity (3a, 3e and 3f) to kill Epstein-Barr virus transformed human B-cells, which are dependent on NF-kB for survival was compared (Cahir-McFarland E D, Carter K, Rosenwald A, et al. Role of NF-kB in cell survival and transcription of latent membrane protein 1-expressing or Epstein-Barr virus latency III-infected cells. J Virol 2004; 78: 4108-4119). ARH77 cells were treated with compounds for 20 hours and cell viability analysed by determining the proportion of cells that did not exclude PI, i.e., had lost plasma membrane integrity (see FIG. 4a for representative experiment). Dose response experiments were performed to determine IC₅₀ values (FIG. 4b). SFZ induced cell death with an IC₅₀ of 1.102±0.250 mM (mean of two determinations ±SD; FIG. 4c). Consistent with their enhanced NF-kB-inhibiting activity, the analogues were all more effective inducers of cell killing than SFZ. Compound 3e was the most effective compound with an IC₅₀ of 0.1954±0.038 mM, whereas compounds 3a and 3f had IC₅₀ values of 0.499±0.093 and 0.407±0.011 mM, respectively.

Induction of Apotosis in CLL and HSC

The ability of SFZ and the analogues to induce CLL apoptosis was analysed. CLL cells from 3 individual patients were treated with SFZ for 6 hours and apoptosis was quantified by analysing PARP cleavage. SFZ induced dose dependent apoptosis in each sample although the extent of apoptosis differed between individual patient samples. The amount of apoptosis induced by SFZ and compounds 3a and 3f (at 0.5 mM) was compared. Compounds 3a and 3f induced more PARP cleavage than SFZ in all three samples (FIG. 5a).

In addition to its role in malignant cells, NF-kB is required for survival of HSC, which play a key role in the pathogenesis of chronic liver disease. The ability of SFZ and analogues to induce HSC apoptosis was also compared. SFZ (1 mM) increased apoptosis ˜4.5-fold in cultured rat HSC (FIG. 5b). Importantly, approximately the same degree of apoptosis was achieved when HSC were treated with compounds 3a, 3e and 3f at approximately 5-fold lower concentrations (i.e., at a concentration that resulted in 50% inhibition of NF-kB activity transcriptional activity). Therefore, the SFZ analogues with improved NF-kB-inhibiting activity are also relatively potent inducers of HSC apoptosis.

Cytotoxicity

Reduction of SFZ in the gut to give SPY is thought to be responsible for much of the toxicity associated with therapy. The putative reduction products of the novel SFZ derivatives were associated with increased or decreased toxicity was investigated. ARH77 cells were treated for 4 days with compounds 2a (i.e., putative reduction product of 3a and 3b), 2c (putative reduction product of 3d), and 2d (putative reduction product of 3e and 3f), and SPY (reduction product of SFZ and 3c). Cell growth was determined using an MTS assay. The percent growth inhibition at 2 mM (FIG. 6) was measured. SPY inhibited the growth of ARH77 cells by 3447% (mean of two determinations ±SD). Cytotoxicity of 2a and 2c was slightly less than SPY with 27±8% and 24±3% growth inhibition, respectively. 2d was essentially non-toxic in this assay with −6±16% growth inhibition. The ability of compounds 2a, 2c and 2d to interfere with NF-kB activity in LPS treated U937-kBluc cells was also tested. None of the compounds significantly inhibited NF-kB activity.

The present results show that substitution of the 5-ASA moiety is tolerated (e.g. in 3c and 3e), without loss of activity against NF-kB. Compounds with improved NF-kB inhibiting activity were also more effective inducers of apoptosis in CLL and HSC. NF-kB is frequently expressed at relatively high levels in CLL and several studies have suggested that NF-kB plays an important role in promoting CLL survival. From the present results, it is shown that pharmacological inhibition of NF-kB is sufficient to accelerate apoptosis in CLL cells cultured ex vivo. The observation that the present analogues that were more effective inhibitors of NF-kB transcriptional activity were also more effective inducers of apoptosis confirms the importance of NF-kB for survival in these systems.

The results show that it is possible to enhance NF-kB activity of SFZ analogues (3e and 3f), while simultaneously decreasing toxicity of the putative reduction product.

In an attempt to understand the basis for the improved activity of the present analogues, the logP values for SFZ, SPY and the analogues have been predicted and compared the values to the IC₅₀ value for each compound for inhibition of NF-kB activity in LPS treated U937 cells (FIG. 7). There is a general trend for improved activity with increased logP. Thus, whereas SFZ had a predicted logP of 2.86, the most active compounds, 3e and 3f had predicted logP values >4. Compounds 3c and 3d, with IC₅₀ values for NF-kB inhibition essentially identical to SFZ, had similar logP values (3.19 and 2.04, respectively). Thus, it is possible that the increased inhibitory activity of 3e and 3f derives, at least in part, from increased hydrophobicity enabling more efficient cellular uptake. However, the logP for 3a (3.29) was not very different from SFZ or 3c, suggesting that the improved inhibitory activity of this compound (7-fold) does not derive from improved cellular uptake. Although the molecular basis for inhibition of IKK is unknown, it is hypothesised that, like other kinase inhibitors, these compounds may bind the ATP binding pocket of IKK molecules. It is possible that the addition of an additional C—C bond and MeO— group allows compound 3a to bind more tightly to the IKK catalytic site. Conversely, the predicted logP for 3b is 3.67 although this compound is not effective at inhibiting NF-kB activity (IC₅₀>2 mM). Therefore, removal of the COOH group in 3c appears to inactivate the molecule rather than effect cell uptake.

### DESCRIPTION OF THE FIGURES

FIG. 1. Inhibition of NF-kB Activity by Sulfasalazine Analogues

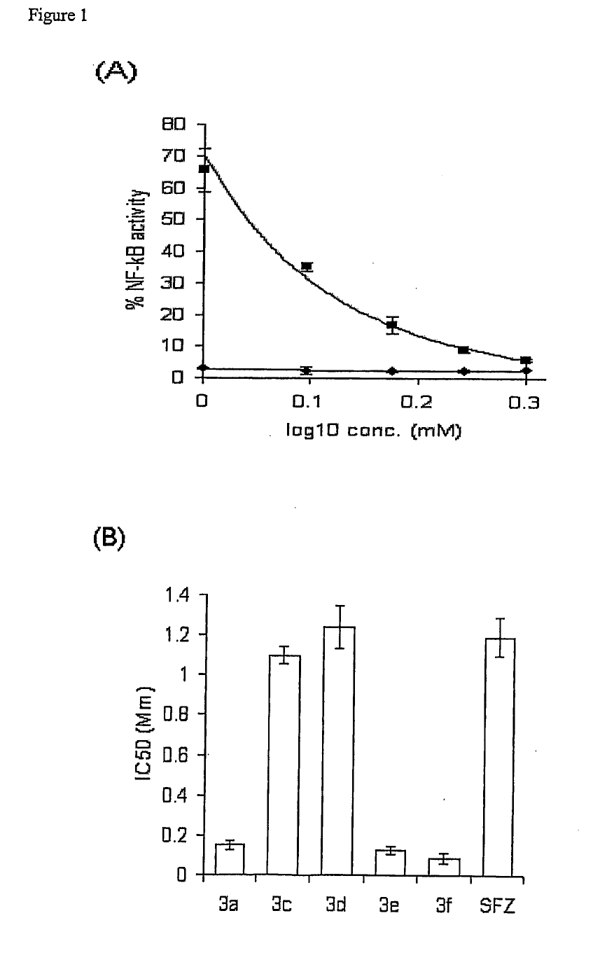

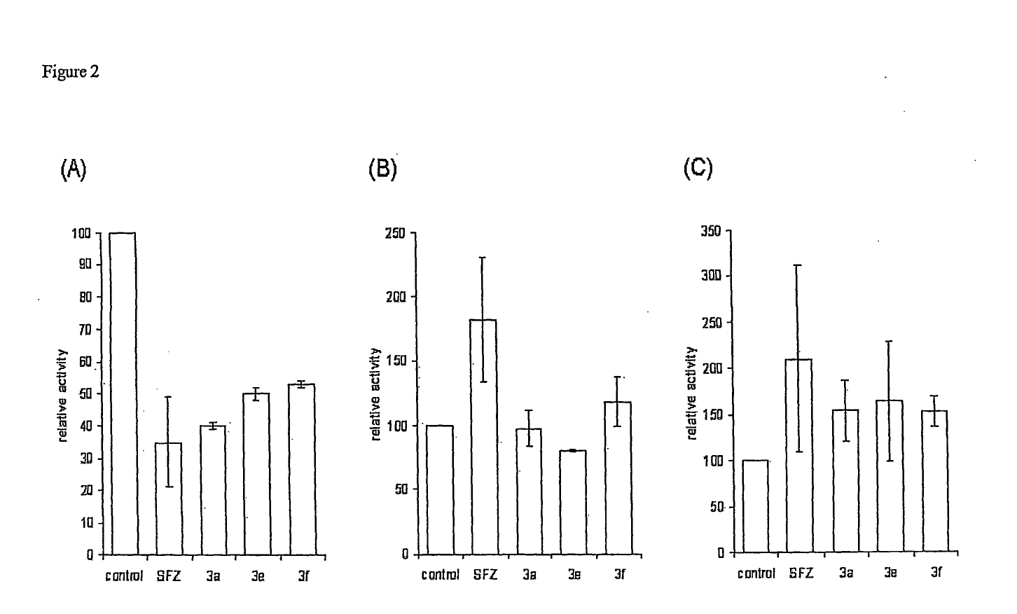

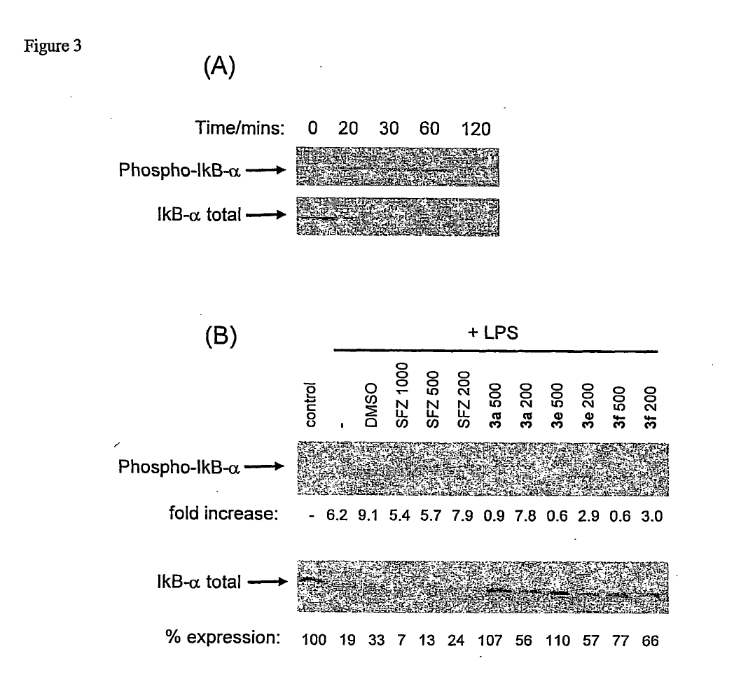

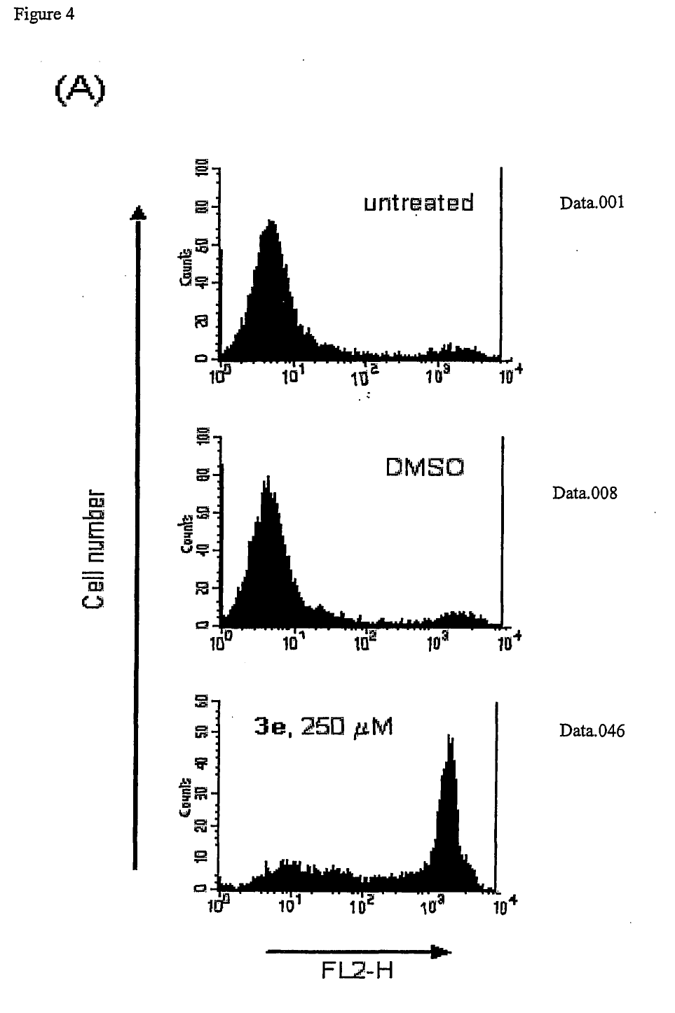

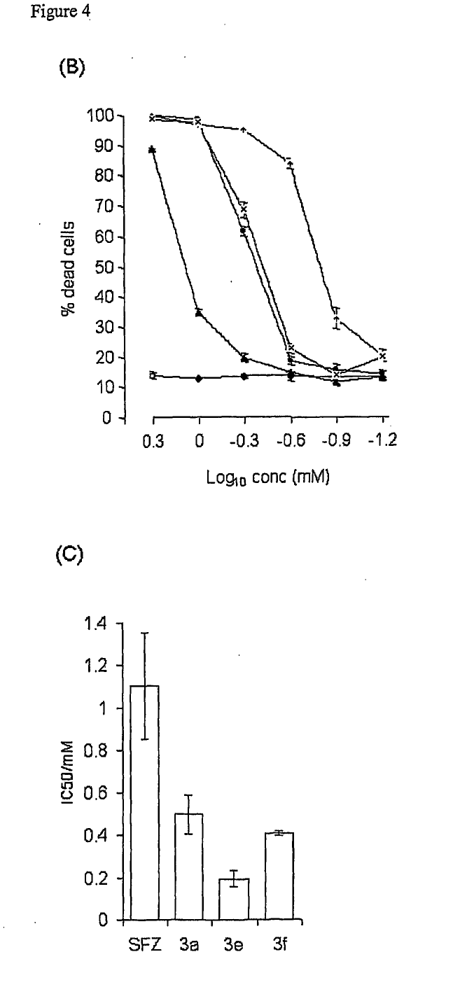

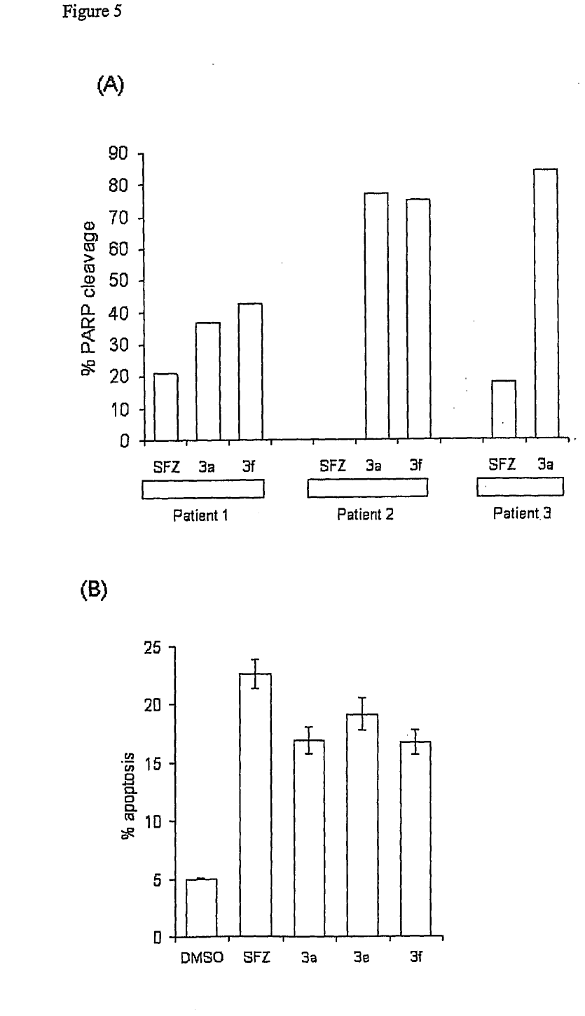

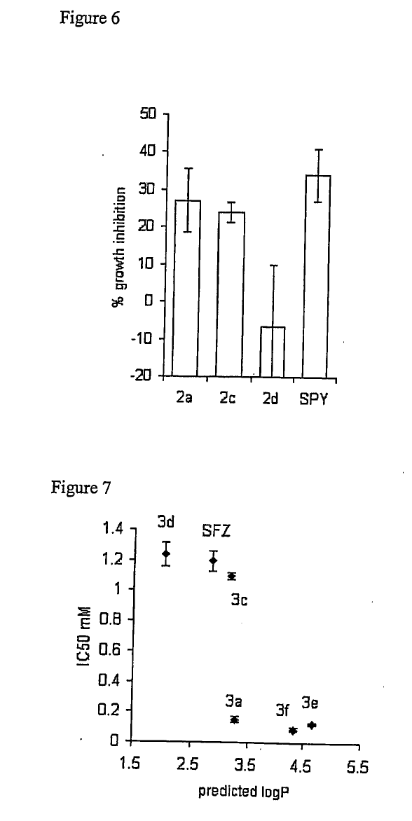

U937-kBluc cells were treated with LPS to activate NF-kB in the presence of increasing concentrations of SFZ, derivatives (3a-f), or DMSO as a solvent control. For each concentration of compound, the percentage inhibition of luciferase activity relative to cells treated with an equivalent amount of DMSO was calculated. (A) Representative dose response experiment (♦, 3a; ▪, SFZ). Each data point is the mean of triplicate samples (±SEM). (B) Detailed dose response assays were performed for compounds which gave >50% inhibition at 2 mM. The mean IC₅₀ value (±SD) for inhibition of LPS-induced NF-kB activity is derived from 2 independent experiments, each performed in triplicate. The average, luciferase activity (±SD) in cells treated with 3b, SPY or 5-ASA (each 2 mM) was 87.5±3.3%, 151±71% and 121±32% of the activity in DMSO treated cells, respectively, and were therefore considered inactive.

FIG. 2. Regulation of Promoter Activity in HSC

Rat HSC were transfected with (A) IkB-α-Luc, (B) TIMP1-Luc or (C) pAP-1-Luc reporter constructs. After 24 hours, cells were treated with sulfasalazine (1 mM), 3a (0.150 mM), 3e (0.127 mM), 3f (0.089 mM), or treated with DMSO as a control. Luciferase activity was determined after another 24 hours. Data presented are mean luciferase activity (±SEM) relative to control cells (value set to 100), derived from two separate experiments, each performed in duplicate and using an independent preparation of HSC.

FIG. 3. IkB-α Phosphorylation and Degradation

(A) U937-kBluc cells were treated with LPS for the indicated times and expression of total IkB-α and phosphorylated IkB-α determined by immunoblotting. (B) U937-kBluc cells were pretreated with the indicated concentrations of compounds or left untreated. After 1 hour, cells were stimulated with LPS. Control cells were cultured in the absence of LPS. Cells were harvested after 30 minutes and the expression of total IkB-α and phosphorylated IkB-α determined by immunoblotting. The signals were quantified by digital image analysis and the fold increase in IkB-α phosphorylation and percent expression of total IkB-α relative to control cells is shown.

FIG. 4. Cell Killing by SFZ Analogues in ARH77 Cells

ARH77 cells were incubated with various concentrations of SFZ, compounds 3a, 3e and 3f, DMSO as a solvent control, or were left untreated. After 20 hours, the proportion of cells that did not exclude PI was determined by flow cytometry as a measure of cell viability. (A) Representative histogram of fluorescence in untreated, DMSO or compound 3e (250 μM) treated cells. (B) Representative dose response experiment (□, untreated; ♦, DMSO; ▴, SFZ; ●, 3a; +, 3e; ×, 3f). Each data point is the mean of duplicate determinations (±SD). (C) Mean IC₅₀ values (±SD) for induction of ARH77 cell death, derived from two independent experiments each performed in duplicate.

FIG. 5. Effect of SFZ Analogues on CLL and HSC Apoptosis

(A) CLL cells from three different patients were cultured in the presence of SFZ or compounds 3a and 3f (all at 0.5 mM), or DMSO as a solvent control. After 6 hours, the degree of PARP cleavage was determined as a measure of apoptosis. The graph shows the increase in PARP cleavage compared to control cells at 6 hours. Note that compound 3f was not tested in cells from patient 3. (B) HSC were cultured in the presence of SFZ (1 mM), compounds 3a (0.150 mM), 3e (0.127 mM), 3f (0.089 mM), or DMSO as a solvent control. Analogues were used at the concentration that gave 50% inhibition of NF-kB transcriptional activity in U937 cells. After 20 hours, the proportion of apoptotic cells were visualised by acridine orange staining. Apoptotic cells were counted in five fields in duplicate for each concentration, in two independent experiments and results expressed as mean ±SEM,

FIG. 6. Cytotoxicity of Putative Reduction Products

ARH77 cells were incubated with 2 mM of 2a, 2c, 2d, SPY or DMSO as a solvent control. After 4 days, cell growth was determined using an MTS assay. Percentage growth inhibition for each compound relative to DMSO treated cells was determined. Mean growth inhibition is shown (±SD). Data are derived from two separate experiments, each performed in triplicate.

FIG. 7. Relationship Between NF-kB Inhibitory Activity and logP

logP values for SFZ and its analogues were calculated using software available from molinspiration, and were compared to IC₅₀ values for inhibition of NF-kB activity in U937 cells. Compound 3b and 5-ASA (which have IC₅₀ values >2 mM) had predicted logP values of 3.667 and 0.664.

1-42. (canceled)

43. A method of treating a patient in need of an NF-kB inhibitor, which method comprises administering to said patient an effective amount of a compound of formula (I), or a pharmaceutically acceptable salt thereof, 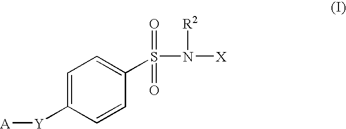
wherein:
A has the following structure; 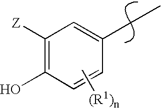
Z is —COOH, —P(O)(OH)₂ or —SO₂OH
each R¹ is the same or different and is halogen, hydroxy, C₁₋₆ alkyl, C₂₋₆ alkenyl, C₂₋₆ alkynyl, C₁₋₆ alkoxy, C₁₋₆ alkylthio, thio, amino, mono(C₁₋₆ alkyl)amino, di(C₁₋₆ alkyl)amino, nitro, cyano or —CO₂R′, wherein R′ represents hydrogen or C₁₋₆ alkyl;
n is 0, 1, 2 or 3;
R² is hydrogen, C₁₋₆ alkyl, C₂₋₆ alkenyl or C₂₋₆ alkynyl;
Y is a linking group; and
X is C₁₋₆ alkyl, C₂₋₆ alkenyl, C₂₋₆ alkynyl, aryl, heteroaryl, carbocyclyl, heterocyclyl, -M-aryl, -M-heteroaryl, -M-carbocyclyl or -M-heterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)—, wherein R′ is as defined above,
provided that:
when n is 0, Z is —COOH, R² is hydrogen and Y is —N═N—, X is other than 2-pyridyl.

44. A method according to claim 43, wherein in formula (I) Z is —COOH.

45. A method according to claim 43, wherein in formula (I) n is 1 or 2.

46. A method according to claim 43, wherein in formula (I) each R¹ is the same or different and is C₁₋₆ alkyl.

47. A method according to claim 43, wherein in formula (I) n is 1, 2 or 3 and one R¹ group is positioned meta to the Y group.

48. A method according to claim 47, wherein in formula (I) the R¹ group positioned meta to the Y group is methyl.

49. A method according to claim 43, wherein in formula (I) A is 2-hydroxy-3-methyl-5-yl-benzoic acid.

50. A method according to claim 43, wherein in formula (I) R² is hydrogen.

51. A method according to claim 43, wherein in formula (I)
Y is —N═N— or C₁₋₆ alkylene, C₂₋₆ alkenylene or C₂₋₆ alkynylene, —C(O)—NR′—, —NR′—C(O)—, -L-Het-L′-, -L-Het- or -Het-L′-;
L and L′ are the same or different and are C₁₋₆ alkylene, C₂₋₆ alkenylene or C₂₋₆ alkynylene; and
Het is selected from —NR′—, —O—, —S—, —SO₂—, —SO—, —C(O)—O—, —OC(O), —CO—, —C(O)—NR′— or —NR′—C(O)— wherein R′ represents hydrogen or C₁₋₆ alkyl.

52. A method according to claim 51, wherein in formula (I) Y is —N═N— or C₁₋₃ alkylene, C₂₋₃ alkenylene, C₂₋₃ alkynylene, -L-Het- or -Het-L′-.

53. A method according to claim 43, wherein in formula (I) Y is —N═N—.

54. A method according to claim 43, wherein in formula (I) X is C₁₋₆ alkyl, aryl, heteroaryl, -M-aryl or -M-heteroaryl.

55. A method according to claim 43, wherein in formula (I) M is C₁₋₆ alkylene.

56. A method according to claim 43, wherein in formula (I) X is pyridyl, pyrazinyl, pyrimidinyl, pyridazinyl, isoxazolyl or benzyl.

57. A method according to claim 43, wherein in formula (I) X is 2-pyridyl, p-methoxy benzyl or p-trifluoromethyl benzyl.

58. A method according to claim 43, wherein the compound of formula (I) is a compound of formula (Ia) 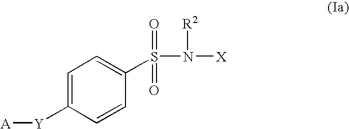
wherein:
A has the following structure; 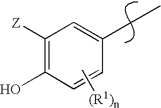
Z, R¹, R², and n;
Y is —N═N—; and
X is -M-aryl, -M-heteroaryl, -M-carbocyclyl or -M-heterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene, Het is selected from —NR′—, —O—, —S—, —SO₂—, —SO—, —C(O)—O—, —OC(O), —CO—, —C(O)—NR′— or —NR′—C(O)— and R′ represents hydrogen or C₁₋₆ alkyl.

59. A method according to claim 58, wherein in formula (Ia) X is -M-aryl or -M-heteroaryl.

60. A method according to claim 58, wherein in formula (Ia) M is C₁₋₆ alkylene.

61. A method according to claim 58, wherein in formula (Ia) X is p-methoxy benzyl or p-trifluoromethylbenzyl.

62. A method according to claim 43, wherein the compound of formula (I) is a compound of formula (Ib) 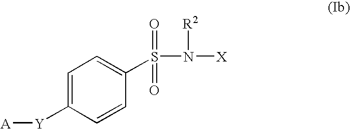
wherein:
A has the following structure; 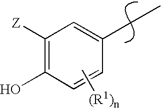
Z, R¹, R² and n;
Y is C₁₋₆ alkylene, C₂₋₆ alkenylene or C₂₋₆ alkynylene, —C(O)—NR′—, -L-Het-L′-, -L-Het- or -Het-L′-, wherein L and L′ are the same or different and are selected from C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ represents hydrogen or C₁₋₆ alkyl; and
X is C₁₋₆ alkyl, C₂₋₆ alkenyl, C₂₋₆ alkynyl, aryl, carbocyclyl, -M-aryl, -M-heteroaryl, -M-carbocyclyl or -M-heterocyclyl, wherein M is C₁₋₆ alkylene, C₂₋₆ alkenylene, C₂₋₆ alkynylene, -Q-Het-Q′- or -Q-Het- wherein Q and Q′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ represents hydrogen or C₁₋₆ alkyl.

63. A method according to claim 62, wherein in formula (Ib) X is -M-aryl or -M-heteroaryl.

64. A method according to claim 62, wherein in formula (Ib) M is C₁₋₆ alkylene.

65. A method according to claim 62, wherein in formula (Ib) Y is C₁₋₃ alkylene, C₂₋₃ alkenylene, C₂₋₃ alkynylene, -L-Het or -Het-L′- wherein Het is —CO—, —NR′— or —C(O)—NR′—.

66. A method according to claim 43, wherein the compound of formula (I) is selected from:
2-hydroxy-5-[4-(4-methoxy-benzylsulfamoyl)-phenylazo]-benzoic acid;
2-hydroxy-3-methyl-5-[4-(pyridin-2-ylsulfamoyl)-phenylazo]benzoic acid;
2-hydroxy-5-(4-methylsulfamoyl-phenylazo)-benzoic acid;
2-hydroxy-3-methyl-5-[4-(4-trifluoromethyl-benzylsulfamoyl)-phenylazo]-benzoic acid; and
2-hydroxy-5-[4-(4-trifluoromethyl-benzylsulfamoyl)-phenylazo]-benzoic acid.

67. A method according to claim 43, wherein the patient is suffering from a condition selected from fibrosis, infection, cancer, inflammatory conditions, stroke, myocardial infarction and reperfusion injury.

68. A method according to claim 67, wherein the condition is fibrosis of the liver.

69. A method according to claim 67, wherein the method is for treating B cell malignant tumours.

70. A method according to claim 43, wherein the compound of formula (I) is comprised in a medicament, which medicament further comprises:
one or more cytotoxic drugs;
an anti-viral drug; or
an immunosuppressant drug.

71. A method of reducing the side effects of a therapy in a patient, wherein the said therapy increases NF-kB activity, which method comprises administering to said patient an effective amount of a compound of formula (I) as defined in claim 43, or a pharmaceutically acceptable salt thereof.

72. A method according to claim 71 wherein the therapy is selected from:
a cytotoxic drug;
radiation therapy;
an anti-viral drug; or
an immunosuppressant drug.

73. A method of reducing, reverting or preventing the development of resistance to a therapy in a patient, wherein the said therapy is rendered less active when NF-kB activity is increased, which method comprises administering to said patient an effective amount of a compound of formula (I) as defined in claim 43, or a pharmaceutically acceptable salt thereof.

74. A method according to claim 73, wherein the therapy is selected from:
a cytotoxic drug;
radiation therapy;
an anti-viral drug; or
an immunosuppressant drug.

75. A method of reducing, reverting or preventing the development of resistance to a therapy in a patient, wherein the said therapy is rendered less active in the presence of NF-kB, which method comprises administering to said patient an effective amount of a compound of formula (I) as defined in claim 43, or a pharmaceutically acceptable salt thereof.

76. A method according to claim 75, wherein the said therapy is selected from:
a cytotoxic drug;
radiation therapy;
an anti-viral drug; or
an immunosuppressant drug.

77. Compound of formula (Ia) as defined in claim 58, or a pharmaceutically acceptable salt thereof.

78. Compound of formula (Ib) as defined in claim 62, or a pharmaceutically acceptable salt thereof.

79. 2-hydroxy-5-[4-(4-methoxy-benzylsulfamoyl)-phenylazo]-benzoic acid;
2-hydroxy-5-(4-methylsulfamoyl-phenylazo)-benzoic acid;
2-hydroxy-3-methyl-5-[4-(4-trifluoromethyl-benzylsulfamoyl)-phenylazo]-benzoic acid; and
2-hydroxy-5-[4-(4-trifluoromethyl-benzylsulfamoyl)-phenylazo]-benzoic acid, and their pharmaceutically acceptable salts.

80. Compound of formula (II), or a pharmaceutically acceptable salt thereof: 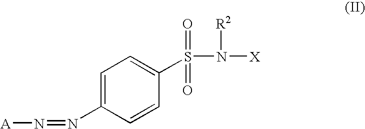
wherein:
A has the following structure 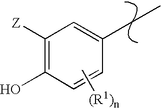
Z, R¹, R² and X are as defined for formula (I) according to claim 43;
n is 1, 2 or 3,
provided that:
when A is 2-hydroxy-3-methyl-5-yl benzoic acid and R² is hydrogen, X is other than 2-pyridyl; and
when A is 2-hydroxy-4-amino-5-yl benzoic acid and R² is hydrogen, X is other than 2-pyridyl.

81. Compound of formula (III), or a pharmaceutically acceptable salt thereof: 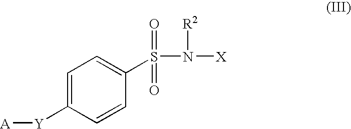
wherein:
A has the following structure 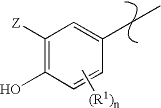
Z, R¹, R² and X are as defined for formula (I) according to claim 43;
Y is C₁₋₆ alkylene, C₂₋₆ alkenylene, C₂₋₆ alkynylene, —C(O)—NR′—, -L-Het-L′-, -L-Het- or -Het-L′-, wherein L and L′ are the same or different and are C₁₋₆ alkylene, C₂₋₆ alkenylene or C₂₋₆ alkynylene and Het is selected from —NR′—, —O—, —S—, —SO₂—, —SO—, —C(O)—O—, —OC(O), —CO—, —C(O)—NR′— or —NR′—C(O)— wherein R′ represents hydrogen or C₁₋₆ alkyl;
n is 1, 2 or 3;
other than 4-fluoro-2-hydroxy-5-[2-[4-[(3-methyl-2-pyridinylamino)sulfonyl]phenyl]ethenyl]benzoic acid.

82. Composition comprising a compound according to claim 79 and a pharmaceutically acceptable carrier.

83. Composition comprising a compound according to claim 80 and a pharmaceutically acceptable carrier.

84. Composition comprising a compound according to claim 81 and a pharmaceutically acceptable carrier.
